(19) United States
(12) Patent Application Publication
(10) Pub. No.: US 2026/0102484 A1
(43) Pub. Date: Apr. 16, 2026

(54) ADJUVANTS FOR ENHANCING THE IMMUNE RESPONSE

(71) Applicants: Lankenau Institute for Medical Research, Wynnewood, PA (US); The Regents of the University of California, Oakland, CA (US)

(72) Inventors: Ellen Heber-Katz, Philadelphia, PA (US); Sam Bollinger, Palo Alto, CA (US); Benjamin Cameron, Philadelphia, PA (US); Phillip B. Messersmith, Richmond, CA (US)

(73) Assignees: Lankenau Institute for Medical Research, Wynnewood, PA (US); The Regents of the University of California, Oakland, CA (US)

(21) Appl. No.: 19/112,442

(22) PCT Filed: Sep. 16, 2023
(86) PCT No.: PCT/US2023/032964

§ 371 Date: Mar. 17, 2025

Related U.S. Application Data
(60) Provisional application No. 63/375,874, filed on Sep. 16, 2022.

(51) Int. Cl. A61K 39/39; A61K 39/00; A61K 39/215; A61K 47/60

(52) U.S. Cl. CPC A61K 39/39; A61K 39/215; A61K 47/60; A61K 2039/55; A61K 2039/55555

(57) ABSTRACT

Compositions and methods enhancing a patient's immune response to an immune stimulatory composition are disclosed. In certain embodiments, the method includes administering a composition comprising a PHD pathway inhibitor and a vaccine to a subject.

### CROSS-REFERENCE TO RELATED APPLICATIONS

This application claims prioirty of U.S. Provisional Application number 63/375,874, filed Sep. 16, 2022, the entire contents being incorporated herein by reference as though set forth in full.

### BACKGROUND OF THE INVENTION

The components of a strong immune response include both arms of an immune response with antibody and T cells. Different viruses have different needs in terms of these responses. It is becoming increasingly clear that potent and long-lived protective immunity against many viruses, such as SARS-CoV-2, may require a robust T cell response.

Substances termed adjuvants that enhance the immune response to vaccines and other immune stimulatory compositions are important elements in effective prophylaxis against infectious diseases, and possibly other more recently investigated diseases such as cancer, infections, and other maladies.

There are relatively few immune adjuvants that have been discovered. Existing adjuvants include alum, monophosphoryl lipid A (MRL), bacterial or viral protein nanoparticles, cytokines, saponin, and dried M. tuberculosis (i.e., Freund's adjuvant, no longer approved for human use). These adjuvants act through known and/or suspected mechanisms including through the creation of antigen depots or directly stimulating receptors on T-cells, B-cells or dendritic cells (e.g., Toll-like receptors [TLRs]).

Antibody responses normally are more protective but that might not be true of certain infections, such as COVID. Sometimes antibodies can be viral protective, but the present vaccines do not have long-lived antibody responses nor T cell responses and are therefore not highly protective. In fact, in these studies, it was shown that little or no T cell reactivity was seen in 5 out of 6 subjects immunized with a COVID vaccine.

Accordingly, there is a clear need for adjuvants that enhance the immune response and increase vaccine protection.

### SUMMARY OF THE INVENTION

The present invention comprises methods for enhancing a patient's immune response to an immune stimulatory composition, the methods comprising administering an immune stimulatory composition and at least one agent that affects metabolic reprogramming. In certain embodiments, the agent that affects metabolic reprogramming is selected from: a) an inhibitor of the proline hydroxylase (PHD) pathway; b) an inhibitor of a p21 kinase; or c) an agonist of HIF-1α. In certain embodiments the agonist of HIF-1α is a modulator of a protein in the HIF regulatory pathway. Also, the agent may at least transiently upregulates, increases, or stabilizes HIF1.

In certain embodiments, the agent is a small molecule, a protein, peptide, or nucleic acid sequence. In embodiments where the agent is a nucleic acid sequence, the nucleic acid sequence may be an siRNA or miRNA.

In certain aspects of the invention, the agent is a PHD inhibitor or prodrug thereof. In certain embodiments the PHD inhibitor is 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), or Hydralazine. In certain embodiments, the PHD inhibitor is 1,4-DPCA.

Another aspect of the invention comprises methods of treating cancer, the methods comprising administering a PHD inhibitor with an anticancer therapy. In certain embodiments the anticancer therapy has vaccine-like effects. In certain embodiments, the anticancer therapy is selected from chemotherapy, radiotherapy, cryotherapy, or another tumor ablative modality.

Also provided herein are methods of enhancing an immune response, the methods comprising administering a PHD inhibitor in combination with a vaccine, wherein the treatment increases the strength and/or potency of the immune response when compared to administration of a vaccine without a PHD inhibitor. In certain embodiments, the vaccine is directed towards an infectious disease. In certain embodiments, the vaccine is directed towards a SARS-CoV2 Spike protein epitope. In certain embodiments, the patient is elderly and/or the patient has an attenuated immune response to the immune stimulatory composition alone when compared to a healthy patient.

In certain embodiments of the invention, the immune stimulatory composition and/or agent are administered in a liposomal formulation. Alternatively, the immune stimulatory composition and/or agent may be administered in a lipid nanoparticle formulation. In certain embodiments, the immune stimulatory composition and/or agent are administered via a subcutaneous or intramuscular injection, or orally or topically at a mucosal site. In certain embodiments, the agent is administered at a concentration of 10-20μM.

Also provided herein are compositions comprising a vaccine and an adjuvant selected from at least one agent that affects metabolic reprogramming. In certain embodiments the agent that affects metabolic reprogramming is selected from: a) an inhibitor of the proline hydroxylase (PHD) pathway; b) an inhibitor of a p21 kinase; or c) an agonist of HIF-1α. In certain embodiments the agonist of HIF-la is a modulator of a protein in the HIF regulatory pathway. In certain embodiments the agent at least transiently upregulates, increases, or stabilizes HIF1. The composition of any one of claims 27 to 30, wherein the agent is a small molecule.

In certain embodiments, the agent is a small molecule, a protein, peptide, or nucleic acid sequence. In embodiments where the agent is a nucleic acid sequence, the nucleic acid sequence may be an siRNA or miRNA.

In certain aspects of the invention, the agent is a PHD inhibitor or prodrug thereof. In certain embodiments the PHD inhibitor is 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), or Hydralazine. In certain embodiments, the PHD inhibitor is 1,4-DPCA.

### BRIEF DESCRIPTION OF THE FIGURES

FIG. 1. A synthetic peptide induces long term protection from lethal infection with herpes simplex virus 2. (Watari, Dietzschold, Szokan, Heber-Katz. 1987, J Ex Med 165: 459-470.)

FIG. 2. 1,4-DPCA enhances antigen-specific (RBD peptide #9) Proliferative T cell response.

FIG. 3. Day 3 in vitro cytokine response to RBD Peptide #9.

FIG. 4. Inverse relationship between lymphoid and myeloid cells at higher DPCA dosage.

FIG. 5A-5B. FIG. 5A: Scatter plots showing the number of cells as determined by FACS analysis in control cells and cells treated with 50 μl DPCA. FIG. 5B Scatter plots showing analysis of myeloid cell markers GR1 and CD11b in control vs. DPCA injected mice.

FIG. 6A-6C. Analysis of three populations of T cells including Naïve (FIG. 6A), Central Memory (FIG. 6B), and Effector Memory (FIG. 6C). Number of cells from lymph nodes and spleen were analyzed after administration of 10 ug, 25 ug or no DPCA.

FIG. 7A-7B. FACS analysis of mice immunized with peptide #5-palmitic acid CFA mixture with and without 10 ug of 1,4-DPCA.

FIG. 8: Structure of 1,4-DPCA and exemplary prodrugs of 1,4-DPCA.

### DETAILED DESCRIPTION OF THE INVENTION

Described herein are compositions and uses thereof that include adjuvants for enhancing immune response of an immune stimulatory molecule. In certain embodiments, the tissue regenerative small molecule 1,4-DPCA is the immune adjuvant.

Adjuvants are incorporated into vaccines to enhance and shape the antigen-specific immune response. They can lead to formation of a depot at the site of injection and upregulate cytokines and chemokines leading to increased cellular recruitment at the injury site. Antigens can increase antigen presentation through myeloid cell populations, through activation and maturation of DCs, and can activate inflammasomes.

Described herein are adjuvants, including the molecule 1,4-DPCA, that can lead to metabolic reprogramming and enhanced immune protection including higher protective T cell cytokines, increased numbers of memory T cells, both CD4 and CD8, broader epitope responses, and a greater level of cell migration.

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys. The term does not denote a particular age or sex. Thus, adult and newborn subjects, as well as fetuses, whether male or female, are intended to be covered.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

It is also contemplated that the term “compound” or “compounds” refers to the compounds discussed herein and includes precursors and derivatives of the compounds, including acyl-protected derivatives, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

As used herein, the term “prodrug” refers to a protected or modified form of the compound, which releases the compound after administration to a subject. For example, a compound may carry a protective group or polymer which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the present disclosure. Thus, the term “prodrug” refers to a metabolic precursor of a compound of the present disclosure that is pharmaceutically acceptable. A prodrug may be inactive when administered to a subject in need thereof, but may be converted in vivo to an active compound of the present disclosure. Prodrugs are typically rapidly transformed in vivo to yield the parent compound of the present disclosure, for example, by hydrolysis in blood. The prodrug compound often offers advantages of solubility, tissue compatibility or delayed release in a subject.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, amelioration, or stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

### Agents That Affect Metabolic Reprogramming

Provided herein are agents that affect metabolic reprogramming and, when incorporated into or administered with immune stimulatory compositions, enhance the subject's immune response. As used herein, the term “agent that affects metabolic reprogramming” is used interchangeably with the term “adjuvant”, as these agents may be incorporated into an antigen-containing (or other) vaccine composition for enhanced response.

In certain embodiments, suitable adjuvant agents include those that increase or stabilize hypoxia-inducible factor 1 (HIF1 or HIF 1-alpha), such as prolyl hydroxylase domain (PHD) inhibitors discussed below. Other suitable agents that inhibit or promote protein in the HIF regulatory pathway can lead to transient HIF upregulation or stabilization. See, e.g., MASOUD, GN and LI, W. HIF-1α pathway: role, regulation and intervention for cancer therapy. 2015; Sept; Acta Pharmaceutica Sinica B, 5(5): 3780-389, incorporated herein by reference for a description of the pathway and inhibitors thereof.

By “prolyl hydroxylase domain enzymes” or “PHDs” is meant a family of enzymes that catalyzes the hydroxylation of certain prolyl residues in collagen precursors using molecular oxygen, ferrous ion, ascorbic acid, and a-ketoglutarate. The members of this family include PHD1, PHD2, and PHD3. They are non-heme iron containing dioxygenases that require oxygen and 2-oxoglutarate as co-substrates and iron and ascorbate as cofactors for their enzymatic activity. See, e.g., GUPTA N and Wish JB, Hypoxia-Inducible Factor Prolyl Hydroxylase Inhibitors: A Potential New Treatment for Anemia in Patients With CKD. 2017 June; Am. J. Kidney Dis., 69(6): 815-826 (epub February 2017). One form of a PHD2 enzyme (prolyl 4-hydroxylase) synthesizes 4-hydroxyprolyl residues, while another produces 3-hydroxyprolyl residues. Among PHDs are two long-known collagen proly1-4-hydroxylases (MYLLYHARJU J, Prolyl 4-hydroxylases, the key enzymes of collagen biosynthesis, 2003 March; Matrix Biol. 22(1):15-24), the more recently identified FIH-1 (factor inhibiting HIF), and PHD1-3, asparaginyl and prolyl hydroxylases, responsible for HIF-1α protein hydroxylation.

Certain small molecules useful as agents in the compositions described herein are the “prolyl hydroxylase domain inhibitors” or “PHDi” small molecules that inhibit or stabilize HIF. Among these are Roxadustat (FG-4592; Fibrogen) described in EICHNER, D et al, cited above. Another molecule is Vadadustat (AKB-6548; Akebia) described in MARTIN ER et al, cited above. Another PHD inhibitor is Daprodustat (GSK-1278863; GlaxoSmithKline) and Molidustat (BAY 85-3934; Bayer). These four above-mentioned PHD inhibitors are described and their dosages in clinical trials detailed in GUPTA and Wish JB, 2017, cited above and incorporated by reference herein.

In one embodiment, the PHD inhibitor is 1,4-dihydrophenonthrolin-4-one-3-carboxylic acid (1,4-DPCA). Other PHD inhibitors useful herein are a prodrug of 1,4-DPCA, or a salt of 1,4-DPCA, Imiquimod or CoCl2 described in U.S. patent application publication No. 20150320877, published Nov. 12, 2015, incorporated herein by reference and other documents cited therein. Still other small molecule PHD inhibitors include dimethyloxalylglycine (DMOG; CAS 89464-63-1) and desferrioxamine B, also known as 30-amino-3,14,25-trihydroxy-3,9,14,20, 25-pentaazatriacontane-2,10,13,21,24-pentone, or a salt thereof; CAS 70-51-9 (EDELMAYER, M et al, Effect of prolyl hydroxylase inhibitor-loaded collagen barrier membranes on osteoclastogenesis and osteoblastogenesis. 2017 May; J. Biomater. Appl., 31(10):1370-1379) and ethyl-3,4-dihydroxybenzoate (EDHB) (HARNOSS JM et al, Prolyl Hydroxylase Inhibition Enhances Liver Regeneration Without Induction of Tumor Growth. 2017 April; Ann Surg., 265(4):782-791. Additional inhibitors that are described in the art include Nepicastat (SYN-117) HCl, (R)-Nepicastat HCl Tetrahydropapaverine HCl, and Norlaudanosine H. See, also, MAXWELL PH and Eckardt KU, HIF prolyl hydroxylase inhibitors for the treatment of renal anaemia and beyond. 2016 March; Nat. Rev. Nephrol. 12(3):157-168.

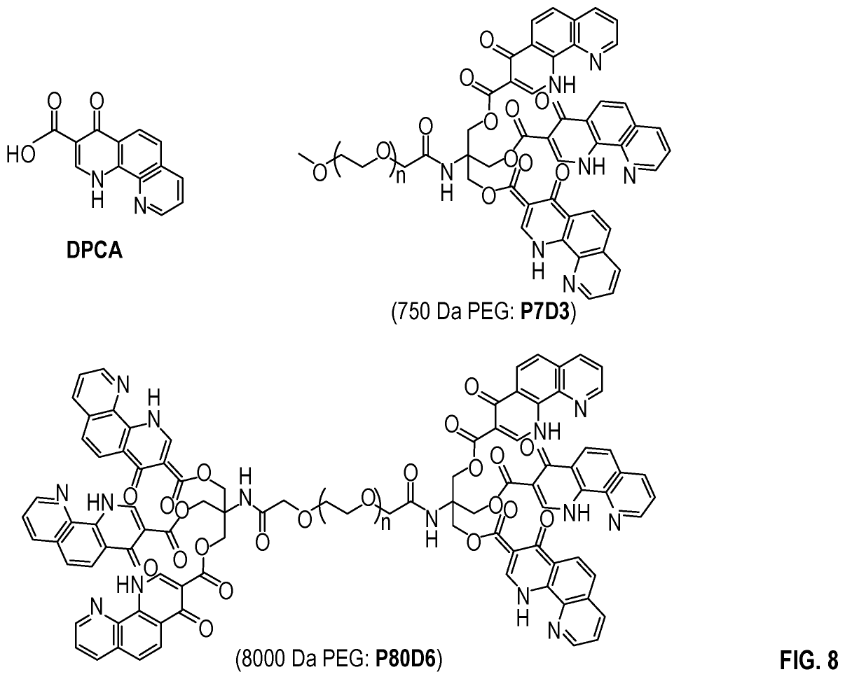

In certain embodiments, the PHD inhibitor is at least one prodrug of 1, 4-DPCA. Various prodrugs of 1, 4-DPCA are known to those skilled in the art and include, without limitation, P7D3 and P80D6 and those described in Cheng J. et al. “Supramolecular Polymer Hydrogels for Drug-Induced Tissue Regeneration” ACS Nano 2019, 13(5), 5493-5501 (incorporated herein by reference). P7D3 comprises three DPCA molecules coupled via a trivalent linker to a 750 Da monomethoxy-PEG. P80D6 comprises a telechelic PEG-DPCA having 6 DPCA molecules coupled via trivalent linkers to a 8000 Da PEG. P7D3 and P80D6 were synthesized from PEG and DPCA using CDI-activated esterification. The structures of P7D3 and P80D6 are shown in FIG. 8.

In certain embodiments the PHD inhibitor comprises at least two prodrugs of 1, 4-DPCA. In certain embodiments, the at least two prodrugs of 1, 4-DPCA comprise a prodrug with a high molecular weight and a prodrug with a low molecular weight. In certain embodiments, the prodrug with a high molecular weight has a higher molecular weight than the prodrug with a low molecular weight. In certain embodiments, the at least two prodrugs of 1,4-DPCA comprise a telechelic prodrug and a monomethoxy prodrug. In certain embodiments the telechelic prodrug has a higher molecular weight than the monomethoxy prodrug. In certain embodiments, the prodrug with a high molecular weight is P80D6. In certain embodiments, the prodrug with a low molecular weight is P7D3. In certain embodiments the percent ratio of prodrug with a higher molecular weight: prodrug with a lower molecular weight in the composition is between approximately 0:100-100:0, 1:99-99:1, 5:95-95:5, 10:90-90:10, 15:85-85:15, 20:80-80:20, 25:75-75:25, 30:70-70:30, 35:65-65:35, 40:60-60:40, 41:59-59:41, 42:58-58:42, 43:57-57:43, 44:56-56:44, 45:55-55:45, 46:54-54:46, 47:53-53:47, 48:52-52-48, 49:51-51:49 or 50:50. In still further embodiments, the ratio of prodrug with a higher molecular weight:prodrug with a lower molecular weight is in any specific ratio or range within these ranges.

In certain embodiments the ratio of prodrug with a higher molecular weight: prodrug with a lower molecular weight is approximately 0:100, 2.5:97.5, 5:95, 7.5:92.5, 10:90, 15:85, 20:80, 25:75, 38:62, 47:53, 55:45, 59:41, 65:35, 75:25, 85:15, 95:5, or 100:0. In a further embodiment, the ratio of prodrug with a higher molecular weight:prodrug with a lower molecular weight is approximately 47:53. It should be evident that the molecular weight of each polymer prodrug is variable while still preserving the described effect.

Such publicly described PHD inhibitor compounds and molecules and their salts derived from pharmaceutically or physiologically acceptable acids, bases, alkali metals and alkaline earth metals are useful in the methods described herein. Still other PHD inhibitors may be found in the catalogs of various biochemical and pharmaceutical suppliers.

Other suitable adjuvant agents are those that decrease expression of the cyclin-dependent kinase inhibitor p21 or inhibit p21. See, e.g., ABBAS, T and Dutta, A, P21 in cancer: intricate networks and multiple activities, Nat Rev Cancer, 2009 June; 9(6):400-414; and BADALBAEVA, K et al. Lack of p21 expression links cell cycle control and appendage regeneration in mice. 2010, March; Proc Natl Acad Sci, USA, 107:5845.

Some inhibitors of p21 include butyrolactone, sorafenib, UC2288, LLW10, Daprodustat (GSK1278863), Vadadustat (AKB-6548), Molidustat (Bay 85-3934), Roxadustat (FG-4592), Desidustat (also known as ZYAN1) and siRNA to p21 and Mir 17-92. See, e.g., LIU, R et al, Small-molecule inhibitors of p21 as novel therapeutics for chemotherapy-resistant kidney cancer. Future Med Chem, 2013 June; 5(9): 991-994; DIB, J, et al. Mass spectrometric characterization of the hypoxia-inducible factor (HIF) stabilizer drug candidate BAY 85-3934 (molidustat) and its glucuronidated metabolite BAY-348,and their implementation into routine doping controls. 2010, January; Drug Testing and Analysis 9(1): 61-67; EICHNER, D, et al. Implementation of the prolyl hydroxylase inhibitor Roxadustat (FG-4592) and its main metabolites into routine doping controls. 2017 November; Drug Testing and Analysis. 9 (11-12): 1768-1778 (epub. 2017 May); JAIN, M., et al. Pharmacological Characterization of ZYAN1, a Novel Prolyl Hydroxylase Inhibitor for the Treatment of Anemia. 2015, September; Drug Research. 66 (02): 107-112; THEVIS M, et al. Mass spectrometric characterization of a prolyl hydroxylase inhibitor GSK1278863, its bishydroxylated metabolite, and its implementation into routine doping controls. 2016 August; Drug Test Anal. 8(8): 858-63; and MARTIN ER, et al. Clinical Trial of Vadadustat in Patients with Anemia Secondary to Stage 3 or 4 Chronic Kidney Disease. 2017, March; Am J Nephrol. 45(5):380-388.

Still another suitable small molecule agent is Ciclopirox, a molecule having the formula C₁₂H₁₇NO₂, PubChem CID 2749. This agent is a synthetic, broad-spectrum antifungal agent with antibacterial and anti-inflammatory activities. Yet another suitable agent is dibenzoylmethane, or 1,3-Diphenyl-1,3-propanedione, having the formula C₁₅H₁₂O₂, PubChem CID 8433, which is a beta-diketone and an aromatic ketone known to exhibit antimutagenic and anticancer effects. It has a role as an antineoplastic agent, a metabolite and an antimutagen. It is available from public sources, e.g., Sigma-Aldrich. Yet another suitable small molecule for use in the compositions described herein is Deferoxamine (DFO) having the formula C₂₅H₄₈N₆O₈, PubChem CID No. 2973. Deferoxamine is an iron-chelating agent that binds free iron in a stable complex, preventing it from engaging in chemical reactions. Deferoxamine chelates iron from intra-lysosomal ferritin and siderin forming ferrioxamine, a water-soluble chelate excreted by the kidneys and in the feces via the bile. This agent does not readily bind iron from transferrin, hemoglobin, myoglobin or cytochrome. (NCI04). Still another suitable small molecule for use in these compositions is hydralazine (also 1-Hydrazinophthalazine) which has the formula C₈H₈N₄ and is a phthalazine derivative with antihypertensive effects. It is available as a variety of salts from public pharmaceutical sources. Still other small molecules are useful.

In some embodiments, the small-molecule adjuvant compound is provided as physiologically acceptable acids. Physiologically acceptable salts include those derived from inorganic and organic acids. A number of inorganic acids are known in the art and include, without limitation, hydrochloric, hydrobromic, hydroiodic, sulfuric, nitric, and phosphoric acid. A number of organic acids are also known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, mallic, phenylacetic, mandelic, embonic, methanesulfonic, ethanesulfonic, panthenoic, benzenesulfonic, toluenesulfonic, stearic, sulfanilic, alginic, and galacturonic acids. Inhibitor compound salts can be also in the form of esters, carbamates, sulfates, ethers, oximes, carbonates, and other conventional “pro-drug” forms, which, when administered in such form, convert to the active moiety in vivo. In one embodiment, the prodrugs are esters. The compounds discussed herein also encompass “metabolites” which are unique products formed by processing the selected inhibitor compound by the cell or subject. In one embodiment, metabolites are formed in vivo.

Also useful as adjuvant agents described herein are “antisense” nucleotide sequence or a small nucleic acid molecule having a complementarity to the nucleic acid sequence of a selected PHD or p21 target described above. Such an antisense sequence can also function as a PHD inhibitor or p21 inhibitor in the methods described herein, such as a nucleic acid sequence having complementarity to a sense region of the small nucleic acid molecule. For example, in one embodiment the composition comprises a nucleic acid construct comprising a sequence that reduces or suppresses the expression of one of the PHD enzymes, p21 targets or a combination thereof. Additionally, or alternatively, in one embodiment, the composition comprises a PHD-inhibitory short nucleic acid molecule (e.g., siRNA). In one embodiment, the PHD-inhibitory short nucleic acid molecule blocks the PHD2 pathway. Such agents that create a PHD blockade led to p21 downregulation in a manner that is critical for epimorphic regeneration.

In a further embodiment, the PHD-inhibitory short nucleic acid molecule transiently upregulates HIF1 or other molecules involved in unleashing the latent potential for ER in mammals. For example, the inhibiting composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence

Still other adjuvant agents described herein are certain peptides and/or proteins. Such proteins can be antibodies (or antibody fragments) that can bind and thus inhibit the activity of PHD or p21 enzymes or proteins in their respective pathways. In one embodiment, p21 agonists align downstream or in parallel with the PHD pathway which limits ER and hence is unleashed by PHDi or PHD siRNA.

In one embodiment, the additional peptide agents useful in these compositions are HIF modulators/ER agonists, such as protease-activated receptor 1 (PAR 1) agonists. In one embodiment, the agent e includes peptides or proteins that are PAR1 agonists or agonists of the PAR1 pathway and their many components which lead to PHD regulation, can also be used to promote ER. PAR1 is a prototype member of an established protease-activated receptor family, which has activity in thrombosis, hemostasis, vascular biology and tumor biology. It is upregulated in regenerating mice, is upstream from HIF, and can activate the HIF pathway. A PAR1 agonist is the peptide TRLLRNPNDK SEQ ID NO: 1 and/or the protein thrombin. See, e.g., AUSTIN, KM et al, Matrix metalloproteases and PAR1 activation, Blood, 2013 January, 121(3): 431-439. In one embodiment, the agent includes small molecules, peptides, proteins, DNA and RNA sequences that interference the PAR1 pathway and result in increased expression or activity of PAR1. These above-identified adjuvant agents enhance immune response.

### Vaccines

Many different types of vaccine are currently in use and many of these are formulated/combined or administered (concurrently or separately) with adjuvants to improve, augment or modify the nature of the immune response raised against the immune stimulatory (e.g., antigen, mRNA) component of the vaccine. In some cases, an adjuvant is exploited as a means to improve an immune response in a human or animal host. For example, an adjuvant may be used to improve the immune response raised by low (sub-optimal) doses of antigen. In this regard, an adjuvant/adjuvant composition of this invention may be combined with sub-optimal doses of a vaccine or antigen-the adjuvant serving to increase the immune response raised by the sub-optimal antigen dose.

When administered together or with an adjuvant or adjuvant composition described herein, the immune response raised by a sub-optimal vaccine/antigen dose, may be comparable to, or greater than, an immune response raised by administration of an optimal dose of vaccine/antigen alone (i.e., without adjuvant). In certain embodiments, the adjuvant is used to raise the immune response of immunocompromised or elderly patients.

When administered together or with an adjuvant or adjuvant composition as described herein, the immune response raised in the immunocompromised or elderly patient, may be comparable to an immune response raised in a healthy patient. In certain embodiments, an elderly patient is at least 55 years old, at least 60 years old, at least 61, 62, 63, 64, 65, 66, 67, 68, 69, 70, 71, 72, 73, 74, 75, 76, 77, 78, 79, 80, 81, 82, 83, 84, or 85 years old.

Adjuvants are particularly useful when the antigen component of a vaccine is poorly or insufficiently immunogenic. For example, certain proteinaceous antigens, in particular those that contain concealed epitopes or domains, which mimic certain host (or self) peptides and/or protein domains, may exhibit an insufficient level of immunogenicity when administered. Additionally, antigens that comprise significant amounts of carbohydrate material (or which consist (essentially) of carbohydrate material) might be less immunogenic than antigens, which are more proteinaceous in nature. Where the antigen (carbohydrate in nature or otherwise) is not sufficiently immunogenic in its own right, an adjuvant is used to improve, augment or modify the immune response raised or induced upon administration of the antigen to a host.

As used herein, the term “antigen vaccine composition” or “antigen vaccine” includes at least one antigen or immunogen in a pharmaceutically acceptable vehicle useful for inducing an immune response in a host. In certain embodiments, the immune stimulatory composition is an antigen vaccine composition.

Antigens that may be used with the adjuvant compositions described herein include viral, parasitic, bacterial or tumor associated antigens. An “antigen” is a molecule or a portion of a molecule capable of being bound by an antibody which is additionally capable of being recognized by, and bound by, an antibody (the corresponding antibody binding region may be referred to as a paratope). In general, epitopes consist of chemically active surface groupings of molecules, for example, amino acids or sugar side chains, and have specific three-dimensional structural characteristics as well as specific charge characteristics. Epitopes are the antigenic determinant on a protein that is recognized by the immune system. The components of the immune system recognizing epitopes are antibodies, T-cells, and B-cells. T-cell epitopes are displayed on the surface of antigen-presenting cells (APCs) and are typically 8-11 (MHC class I) or 15 plus (MHC class II) amino acids in length. Recognition of the displayed MHC-peptide complex by T-cells is critical to their activation. These mechanisms allow for the appropriate recognition of “self” versus “non-self” proteins such as bacteria and viruses. Independent amino acid residues that are not necessarily contiguous contribute to interactions with the APC binding cleft and subsequent recognition by the T-Cell receptor (Janeway, Travers, Walport, Immunobiology: The Immune System in Health and Disease. 5th edition New York: Garland Science; 2001). Epitopes that are recognized by soluble antibodies and cell surface associated B-cell receptors vary greatly in length and degree of continuity (Sivalingam and Shepherd, Immunol. 2012 July; 51(3-4):304-309 9). Again even linear epitopes or epitopes found in a continuous stretch of protein sequence will often have discontiguous amino acids that represent the key points of contact with the antibody paratopes or B-cell receptor. Epitopes recognized by antibodies and B-cells can be conformational with amino acids comprising a common area of contact on the protein in three-dimensional space and are dependent on tertiary and quaternary structural features of the protein. These residues are often found in spatially distinct areas of the primary amino acid sequence.

Antigens that can be included in the vaccine composition include antigens prepared from pathogenic bacteria such as Mycoplasma hyopneumoniae, Haemophilus somnus, Haemophilus parasuis, Bordetella bronchiseptica, Actinobacillus pleuropneumonie, Pasteurella multocida, Manheimia hemolytica, Mycoplasma bovis, Mycoplasma galanacieum, Mycobacterium bovis, Mycobacterium paratuberculosis, Clostridial spp., Streptococcus uberis, Streptococcus suis, Staphylococcus aureus, Erysipelothrix rhusopathiae, Campylobacter spp., Fusobacterium necrophorum, Escherichia coli, Salmonella enterica serovars, Leptospira spp. ; pathogenic fungi such as Candida; protozoa such as Cryptosporidium parvum, Neospora canium, Toxoplasma gondii, Eimeria spp.; helminths such as Ostertagia, Cooperia, Haemonchus, Fasciola, either in the form of an inactivated whole or partial cell preparation, or in the form of antigenic molecules obtained by conventional protein purification, genetic engineering techniques or chemical synthesis. Additional antigens include pathogenic viruses such as Bovine herpesviruses-1,3,6, Bovine viral diarrhea virus (BVDV) types 1 and 2, Bovine parainfluenza virus, Bovine respiratory syncytial virus, bovine leukosis virus, rinderpest virus, foot and mouth disease virus, rabies, swine fever virus, African swine fever virus, Porcine parvovirus, PRRS virus, Porcine circovirus, influenza virus, swine vesicular disease virus, Techen fever virus, Pseudorabies virus, either in the form of an inactivated whole or partial virus preparation, or in the form of antigenic molecules obtained by conventional protein purification, genetic engineering techniques or chemical synthesis.

In one embodiment, the encoded antigen is derived from a virus such as influenza, including inactivated influenza virus or influenza haemagglutinin, neuraminidase or M2 protein or other components of the influenza virus. Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus, the genus Apthovirus (Foot and Mouth disease; the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, FlandersHart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that are antigens in vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesvirises (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D, E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papillomaviruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc). DNA viruses also include Kuru and Creutzfeldt-Jacob disease viruses and chronic infectious neuropathic agents (CHINA virus).

Other examples of suitable antigens include, but are not limited to, infectious disease antigens for which a protective immune response may be desired including the human immunogenicity virus (HIV) antigens gag, env, pol, tat, rev, nef, reverse transcriptase, and other HIV components or a part thereof, the E6 and E7 proteins from human papilloma virus, the EBNA1 antigen from herpes simplex virus, hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin, neuraminidase, nucleoprotein, M2, and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegalovirus antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpl, gpll, and other varicella zoster viral antigen components; Japanese encephalitis viral antigens such as proteins E, M-E, M-E-NS1, NS 1, NS 1-NS2A; rabies viral antigens such as rabies glycoprotein, rabies nucleoprotein and other rabies viral antigen components; West Nile virus prM and E proteins; and Ebola envelope protein. See Fundamental Virology, Second Edition, eds. Knipe, D. M. and, Howley P. M. (Lippincott Williams & Wilkins, New York, 2001) for additional examples of viral antigens.

Examples of protozoa and other parasitic antigens include, but are not limited to, plasmodium falciparum antigens such as merozoite surface antigens, sporozoite surface antigens, circumsporozoite antigens, gametocyte/gamete surface antigens, blood-stage antigen pt 1 55/RESA and other plasmodial antigen components; toxoplasma antigens such as SAG-1, p30 and other toxoplasma antigen components; schistosomae antigens such as glutathione-Stransferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components. Examples of fungal antigens include, but are not limited to, antigens from Candida species, Aspergillus species, Blastomyces species, Histoplasma species, Coccidiodomycosis species, Malassezia furfur and other species, Exophiala werneckii and other species, Piedraia hortai and other species, Trichosporum beigelii and other species, Microsporum species, Trichophyton species, Epidermophyton species, Sporothrix schenckii and other species, Fonsecaea pedrosoi and other species, Wangiella dermatitidis and other species, Pseudallescheria boydii and other species, Madurella grisea and other species, Rhizopus species, Absidia species, and Mucor species. Examples of prion disease antigens include PrP, beta-amyloid, and other prion-associated proteins.

In addition to the infectious and parasitic agents mentioned above, another area for desirable enhanced immunogenicity to a non-infectious agent is in the area of cancer, in which cells expressing cancer antigens are desirably eliminated from the body. A cancer antigen as used herein is a compound, such as a peptide or protein, present in a tumor or cancer cell and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared by recombinant DNA expression technology or by any other means known in the art. In one embodiment, the cancer is selected from the group consisting of biliary tract cancer; bone cancer; brain and CNS cancer; breast cancer; cervical cancer; choriocarcinoma; colon cancer; connective tissue cancer; endometrial cancer; esophageal cancer; eye cancer; gastric cancer; Hodgkin's lymphoma; intraepithelial neoplasms; larynx cancer; lymphomas; liver cancer; lung cancer (e.g. small cell and non-small cell); melanoma; neuroblastomas; oral cavity cancer; ovarian cancer; pancreas cancer; prostate cancer; rectal cancer; sarcomas; skin cancer; testicular cancer; thyroid cancer; and renal cancer. Cancer antigens which can be used in the compositions and methods of the invention include, but are not limited to, prostate specific antigen (PSA), breast, ovarian, testicular, melanoma, telomerase; multidrug resistance proteins such as P-glycoprotein; MAGE-1, alpha fetoprotein, carcinoembryonic antigen, mutant p53, papillomavirus antigens, gangliosides or other carbohydrate-containing components of melanoma or other cancer cells. It is contemplated that antigens from any type of cancer cell can be used in the compositions and methods described herein. The antigen may be a cancer cell, or immunogenic materials isolated from a cancer cell, such as membrane proteins. Included are survivin and telomerase universal antigens and the MAGE family of cancer testis antigens.

In another preferred embodiment, the compositions, substances and methods described herein can be used with antigens known as “allergens” involved in allergy to induce tolerance and suppress allergen-specific IgE. An allergen is any substance that can induce an allergic or asthmatic response in a susceptible subject. Allergens include pollens, insect venoms, animal dander dust, fungal spores and drugs (e.g. penicillin). Examples of natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Parrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In another embodiment, the compositions, substances and methods described herein can be used to immunize against antigens involved in asthma. Such antigens include, but are not limited to, IgE and histamine.

In certain embodiments, the antigen includes a polynucleotide that encodes the polypeptide that functions as the antigen, i.e., a nucleic acid vaccine. As used herein, the term “polynucleotide” encompasses a chain of nucleotides of any length (e.g., 9, 12, 18, 24, 30, 60, 150, 300, 600, 1500 or more nucleotides) or number of strands (e.g., single-stranded or double-stranded). Polynucleotides may be DNA (e.g., genomic DNA or cDNA) or RNA (e.g., mRNA) or combinations thereof. They may be naturally occurring or synthetic (e.g., chemically synthesized). It is contemplated that the polynucleotide may contain modifications of one or more nitrogenous bases, pentose sugars or phosphate groups in the nucleotide chain. Such modifications are well-known in the art and may be for the purpose of e.g., improving stability of the polynucleotide.

The polynucleotide may be delivered in various forms. In some embodiments, a naked polynucleotide may be used, either in linear form, or inserted into a plasmid, such as an expression plasmid. In other embodiments, a live vector such as a viral or bacterial vector may be used.

One or more regulatory sequences that aid in transcription of DNA into RNA and/or translation of RNA into a polypeptide may be present. In some instances, such as in the case of a polynucleotide that is a messenger RNA (mRNA) molecule, regulatory sequences relating to the transcription process (e.g., a promoter) are not required, and protein expression may be effected in the absence of a promoter. The skilled artisan can include suitable regulatory sequences as the circumstances require.

In various embodiments, the composition comprises an in vitro transcribed (IVT) RNA molecule. An mRNA may include a 5′ untranslated region, a 3′ untranslated region, and/or a coding or translating sequence.

An mRNA may be a naturally or non-naturally occurring mRNA. An mRNA may include one or more modified nucleobases, nucleosides, or nucleotides. In some embodiments, the mRNA comprises at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence).

In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids also include the incorporation of pseudouridines pseudouridine (ψ) or 5-methylcytosine (m5C). Substitutions and modifications to the mRNA of the present invention may be performed by methods readily known to one or ordinary skill in the art.

In certain embodiments, the mRNA includes a 5′ cap structure, a chain terminating nucleotide, a stem loop, a polyA sequence, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a poly A sequence. A poly A sequence may be comprised entirely or mostly of adenine nucleotides or analogs or derivatives thereof. In certain embodiments, the poly A sequence is a tail located adjacent to a 3′ untranslated region of an mRNA.

In some embodiments, e.g., in a DNA vaccine, the polynucleotide is present in an expression cassette, in which it is operably linked to regulatory sequences that will permit the polynucleotide to be expressed in the subject to which the composition of the invention is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g., the signal peptide), is placed under the control of the promoter so that transcription and translation occur in the subject to which the composition is administered.

Cancer vaccines are designed to elicit an immune response against tumor-specific or tumor-associated antigens, encouraging the immune system to attack cancer cells bearing these antigens. Cancer vaccines can be made from a variety of components, including cells, proteins, DNA, viruses, bacteria, and small molecules. Cancer vaccine targets under evaluation in multiple myeloma clinical trials include: Melanoma-associated antigen (MAGE): the genes that produce these proteins are normally turned off in adult cells, but can become reactivated in cancer cells, flagging them as abnormal to the immune system; Survivin: a protein that can prevent cellular death and is overexpressed by a number of cancer cell types; Telomerase: an enzyme that helps maintain the health of cellular DNA; exploited by cancer cells to achieve immortality; Tumor-associated antigens (TAAs): proteins often expressed at abnormally high levels on tumor cells that can be used to target them; also found on normal cells at lower levels; WT1: a protein that is often mutated and abnormally expressed in patients with cancer, especially Wilms' tumor (WT).

Vaccine compositions can be administered in dosages, and by techniques well known to those skilled in the medical or veterinary arts, taking into consideration factors such as the age, sex, weight, species and condition of the recipient mammal, and the route of administration. Vaccine compositions can be administered alone, or can be co-administered or sequentially administered with other treatments or therapies, including an adjuvant described herein. In certain embodiments, the vaccine composition is administered in a separate composition as the adjuvant. In other embodiments, the vaccine composition and adjuvant are formulated into a single composition. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences” (1990), may be consulted to prepare suitable preparations, without undue experimentation.

### Methods of Treatment

Provided herein are methods of enhancing an immune response to a vaccine comprising administering at least one agent that blocks or inhibits the proline hydroxylase (PHD) pathway; inhibits or decreases p21; modulates any protein in the HIF regulatory pathway. In certain embodiments the agonist of HIF-la modulates any element in the HIF regulatory pathway. An element in the HIF regulatory pathway can be, without limitation, a microRNA, a metal ion, a protein, RNA, or other nucleic acid. In certain embodiments, the agent at least transiently upregulates, increases, or stabilizes HIF1. The agent may be selected from a small molecule, protein, peptide, or nucleic acid sequence, such as an siRNA or miRNA. In certain embodiments, the agent is a PHD inhibitor. The PHD inhibitor can be selected from 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), or Hydralazine. In certain embodiments, the prodrug is at least one of P7D3 and P80D6 and those described in Cheng J. et al. “Supramolecular Polymer Hydrogels for Drug-Induced Tissue Regeneration” ACS Nano 2019, 13(5), 5493-5501 (incorporated herein by reference). In certain embodiments the PHD inhibitor comprises at least two prodrugs of 1, 4-DPCA. In certain embodiments, the at least two prodrugs of 1, 4-DPCA comprise a prodrug with a high molecular weight and a prodrug with a low molecular weight. In certain embodiments, the prodrug with a high molecular weight is P80D6. In certain embodiments, the prodrug with a low molecular weight is P7D3. In certain embodiments the percent ratio of prodrug with a high molecular weight: prodrug with a low molecular weight in the composition is between approximately 0:100-100:0, 1:99-99:1, 5:95-95:5,10:90-90:10, 15:85-85:15, 20:80-80:20, 25:75-75:25, 30:70-70:30, 35:65-65:35, 40:60-60:40, 41:59-59:41, 42:58-58:42, 43:57-57:43, 44:56-56:44, 45:55-55:45, 46:54-54:46,47:53-53:47, 48:52-52-48, 49:51-51:49 or 50:50. In still further embodiments, the ratio of prodrug with a high molecular weight:prodrug with a low molecular weight is in any specific ratio or range within these ranges.

In certain embodiments the ratio of prodrug with a high molecular weight: prodrug with a low molecular weight is approximately 0:100, 2.5:97.5, 5:95, 7.5:92.5, 10:90, 15:85, 20:80, 25:75, 38:62, 47:53, 55:45, 59:41, 65:35, 75:25, 85:15, 95:5, or 100:0. In a further embodiment, the ratio of prodrug with a high molecular weight: prodrug with a low molecular weight is approximately 47:53.

In certain embodiments, the vaccine is directed towards an infectious disease such as a SARS-CoV2 Spike protein epitope. In certain embodiments, the patient is elderly and/or has an attenuated immune response to the immune stimulatory composition alone when compared to a healthy patient. The immune stimulatory composition may be administered in a liposomal formulation, lipid nanoparticle formulation, subcutaneous injection, intramuscular injection, or orally or topically at a mucosal site.

The immune stimulatory composition may be administered in a liposomal formulation. Various amphiphilic lipids can form bilayers in an aqueous environment to encapsulate a RNA-containing aqueous core as a liposome. These lipids can have an anionic, cationic or zwitterionic hydrophilic head group. Some phospholipids are anionic whereas others are zwitterionic and others are cationic. Suitable classes of phospholipids include, but are not limited to, phosphatidylethanolamines, phosphatidylcholines, phosphatidylserines, and phosphatidyl-glycerols. Useful cationic lipids include, but are not limited to, dioleoyl trimethylammonium propane (DOTAP), 1,2-distearyloxy-N, N-dimethyl-3-aminopropane (DSDMA), 1,2-dioleyloxy-N, N dimethyl-3-aminopropane (DODMA), 1,2-dilinoleyloxy-N, N-dimethyl-3-aminopropane (DLinDMA), 1,2-dilinolenyloxy-N, N-dimethyl-3-aminopropane (DLenDMA). Further useful cationic lipids are described in WO 15/095340, for example the lipids as claimed in any of claims 1 to 8 of WO 15/095340 incorporated herein by reference. Zwitterionic lipids include, but are not limited to, acyl zwitterionic lipids and ether zwitterionic lipids. Examples of useful zwitterionic lipids are DPPC, DOPC and dodecylphosphocholine. Liposomal particles of the invention can be formed from a single lipid or from a mixture of lipids. A mixture may comprise (i) a mixture of anionic lipids, (ii) a mixture of cationic lipids, (iii) a mixture of zwitterionic lipids, (iv) a mixture of anionic lipids and cationic lipids, (v) a mixture of anionic lipids and zwitterionic lipids, (vi) a mixture of zwitterionic lipids and cationic lipids or (vii) a mixture of anionic lipids, cationic lipids and zwitterionic lipids. Where a mixture of lipids is used, not all of the component lipids in the mixture need to be amphiphilic e.g. one or more amphiphilic lipids can be mixed with cholesterol. The hydrophilic portion of a lipid can be PEGylated (i.e. modified by covalent attachment of a polyethylene glycol). This modification can increase stability and prevent non-specific adsorption of the liposomes. Liposomal particles are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter 50 nm, and LUVs have a diameter>50 nm. Liposomal particles useful in this aspect of the invention are ideally LUVs with a diameter in the range of 50-220 nm. Techniques for preparing suitable liposomes are well known in the art. One useful method is described in Jeffs et al. (Pharmaceutical Research, 2005, 22(3): 362-372) and involves mixing (i) an ethanolic solution of the lipids (ii) an aqueous solution of the nucleic acid and (iii) buffer, followed by mixing, equilibration, dilution and purification.

In certain embodiments, the vaccine and/or adjuvant is hydrophobic and sits in the liposome membrane surrounded by lipid moieties. In certain embodiments, the vaccine and/or adjuvant is released upon delivery. In certain embodiments, the vaccine and/or adjuvant is released after the liposome is taken up by the cells.

The immune stimulatory composition may be administered in a lipid nanoparticle formulation. Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid. In some embodiments, the lipid nanoparticle comprises 45-55 mole percent ionizable cationic lipid. For example, lipid nanoparticle may comprise 45, 46, 47, 48, 49, 50, 51, 52, 53, 54, or 55 mole percent ionizable cationic lipid.

In some embodiments, the lipid nanoparticle comprises 5-15 mole percent DSPC. For example, the lipid nanoparticle may comprise 5, 6, 7, 8, 9, 10, 11, 12, 13, 14, or 15 mole percent DSPC.

In some embodiments, the lipid nanoparticle comprises 35-40 mole percent cholesterol. For example, the lipid nanoparticle may comprise 35, 36, 37, 38, 39, or 40 mole percent cholesterol.

In some embodiments, a LNP of the disclosure has a mean diameter from about 50 nm to about 150 nm. In some embodiments, a LNP of the disclosure has a mean diameter from about 70 nm to about 120 nm.

In one or more embodiments, the immunogenic compositions and/or vaccines of the present disclosure may be formulated as an injectable. The immune stimulatory composition may be administered by subcutaneous injection. The immune stimulatory composition may be administered by intramuscular injection. The immune stimulatory composition may be administered orally or topically at a mucosal site.

In certain embodiments, the adjuvant increases the strength and/or potency of the immune response of a vaccine when compared to administration of a vaccine without a PHD inhibitor. In certain embodiments, the strength and/or potency is increased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent or more.

The word “potency” refers to the specific ability or capacity of the vaccine, as indicated by appropriate laboratory tests or by adequately controlled clinical data obtained through the administration of the vaccine in the manner intended, to effect protective immunity. Presently, different test methods, such as assays of physiochemical properties, antigenicity, immunogenicity, infectivity and protection against infection or disease, are used to measure vaccine potency. Their application depends on the nature of the vaccine antigens and the purpose of the test. In live vaccines, potency can be based on the number of organisms present in the vaccine (titre). In the case of inactivated vaccines, the potency is often determined by measuring the immune response in the target animal species or in another species, e.g., mice or rats. Alternatively, the potency of an inactivated vaccine can be based on its antigenicity by measuring the quantity of the antigen present (antigen mass), using immunoassays that employ specific antibodies, such as an ELISA (enzyme-Linked immunosorbent assay).

In another aspect of the invention are methods of treatment of cancer, infections and other malaise via administration of at least one agent that blocks or inhibits the proline hydroxylase (PHD) pathway, inhibits or decreases p21, or modulates any protein in the HIF regulatory pathway. In certain embodiments, the method comprises an anticancer therapy that has vaccine-like effects. As used herein, “vaccine-like effect” refers to therapies that provide enhanced immunoprotection in the subject, such as through strong and long-lasting antiviral humoral and/or cytotoxic T-cell responses. In certain embodiments, anticancer therapy can be selected from chemotherapy, radiotherapy, cryotherapy, or another tumor ablative modality. In certain embodiments, the method of treatment alleviates symptoms of the cancer, infection, or other malaise.

In certain embodiments, the agent at least transiently upregulates, increases, or stabilizes HIF1. The agent may be selected from a small molecule, protein, peptide, or nucleic acid sequence, such as an siRNA or miRNA. In certain embodiments, the agent is a PHD inhibitor. The PHD inhibitor can be selected from 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), Hydralazine, P80D6, or P7D3.

In certain embodiments, the at least one adjuvant and at least one vaccine are administered together or sequentially. By sequential administration, the adjuvant may be delivered to a subject before or after administration of the at least one vaccine. In further embodiments, the adjuvant may be delivered to a subject at least 1, 2, 3, 4, 5, 6, 7, 8, 9, 10, 11, 12, 13, or 14 days, or 1 week, 2 weeks, 3 weeks, 4 weeks, before or after administration of the vaccine. In certain embodiments, the adjuvant is administered at least 1-4 weeks before administration of the vaccine. In still further embodiments, the adjuvant may be delivered to a subject in any combination of months, days, hours, minutes, and seconds within these ranges. In further embodiments, the administration of vaccine and adjuvant may be repeated multiple times.

### Administration

The compounds described herein can be formulated for enteral, parenteral, topical, or cardiac administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

The compounds described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion. Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

In certain embodiments, the adjuvant is delivered with an immune stimulatory component in a liposome formulation. Liposomal formulations of vaccines are known in the art. For example, in 1985, B. Dietzschold and E. Heber-Katz demonstrated the same using HSV-2 glycoprotein D (gD) peptides. The original peptides focused on the N-terminus of the gD. The first 23 N-terminal peptides were potent and induced a strong T cell response. They produced a peptide vaccine and used additional components to enhance its activity. First, two fatty acid chains (determined to be optimal using palmitic acid—16 C) were added to the linker KGG which itself was added to the N terminus of the peptide. The peptide with its hydrophobic tail could then be inserted into a liposome. The liposome was composed of phosphatyl choline, cholesterol, and lysolecithin mixed in a ratio of 16:2:1. From electron micrographs, it was shown that the C14-labeled palmitic acid-peptides were displayed on the outside of the liposomes.

For intravenous administration, the compositions may be packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

The compounds described herein can be administered in an effective amount to a subject that is in need of enhancement of immune response. The compounds described herein can be administered in an effective amount to a subject that is in need of alleviation or amelioration from one or more symptoms associated with cancer, infection disorder.

The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation. The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The compositions are administered in an effective amount and for a period of time effect to reduce one or more symptoms associated with the disease to be treated. It should be understood that the “effective amount” for a composition which comprises a sodium channel blocker, an agent that increases extracellular potassium, or a modification thereof, may vary. In one embodiment an effective amount includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body weight. In another embodiment, the range of effective amount is 40 to 50 mg/kg body weight. In another embodiment, the range of effective amount is 1 to 50 mg/kg body weight. Still other doses falling within these ranges are expected to be useful.

In another embodiment, the range of effective amount is 0.001 mg to 10 g. In another embodiment, the range of effective amount is 0.01 mg to 1 g. In another embodiment, the range of effective amount is 0.01 mg to 100 mg. In another embodiment, the range of effective amount is 0.1 mg to 100 mg. In another embodiment, the range of effective amount is 0.1 mg to 500 mg. In another embodiment, the range of effective amount is 1 mg to 100 mg. In another embodiment, the range of effective amount is 10 mg to 500 mg. In another embodiment, the range of effective amount is 10 mg to 750 mg. In another embodiment, the range of effective amount is 0.01 mg to 100 mg. In another embodiment, the range of effective amount is 1 mg to 500 mg. In another embodiment, the effective amount is 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, or 1000 mg.

### KITS AND ARTICLES OF MANUFACTURE

Any of the aforementioned products can be incorporated into a kit which may contain at least one agent that blocks or inhibits the proline hydroxylase (PHD) pathway, inhibits or decreases p21, or modulates any protein in the HIF regulatory pathway; a vaccine; a pharmaceutically acceptable carrier; instructions for use; a container; a vessel for administration; or any combination thereof.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: 1,4DPCA-PEG Effect on Antigen-Specific T Cell Responses

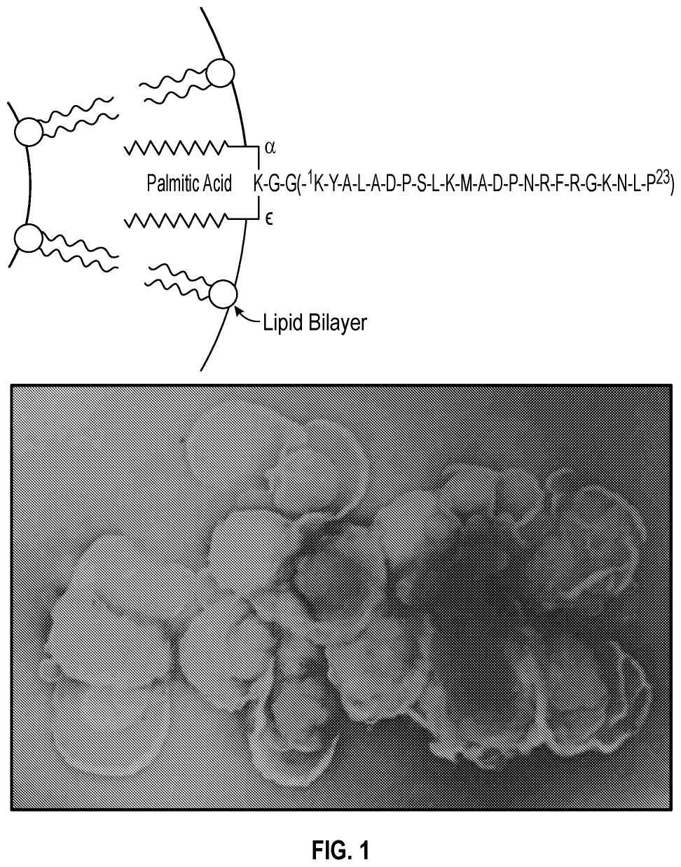

We have used COVID spike peptides to induce an immune response and determine the role of 1,4-DPCA-PEG, drug+a time-release carrier. To determine the response to a peptide, and in this case, a COVID 19 peptide from the Spike protein was administered. We used a peptide from either the N-terminus (peptide #5) or the receptor binding motif (peptide #9) and examined proliferation, cell type, drug dosage and cytokine production. (FIG. 1)

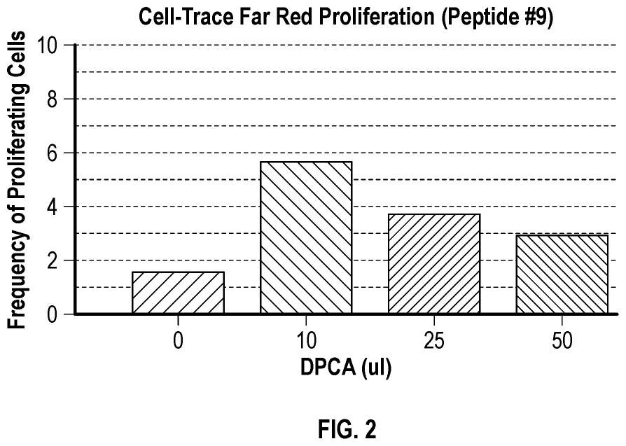

Mice were immunized with peptide antigen #9 on day 0 and given DPCA on the same day. Different doses of drug were given (0, 10, 25, and 50 ug/mouse). Cells from the spleen were cultured with antigen and proliferation determined. (FIG. 2) The drug was given at the same time as the antigen and proliferation was measured on day 3 after stimulation by cell trace (CTFR) levels. Here, 50 ug drug was suppressive. However, 10 ug was most stimulatory. Thus, the dose of DPCA was important.

Next, we examined the effect of the drug on the cytokines produced. The most potent anti-viral response is INFg.

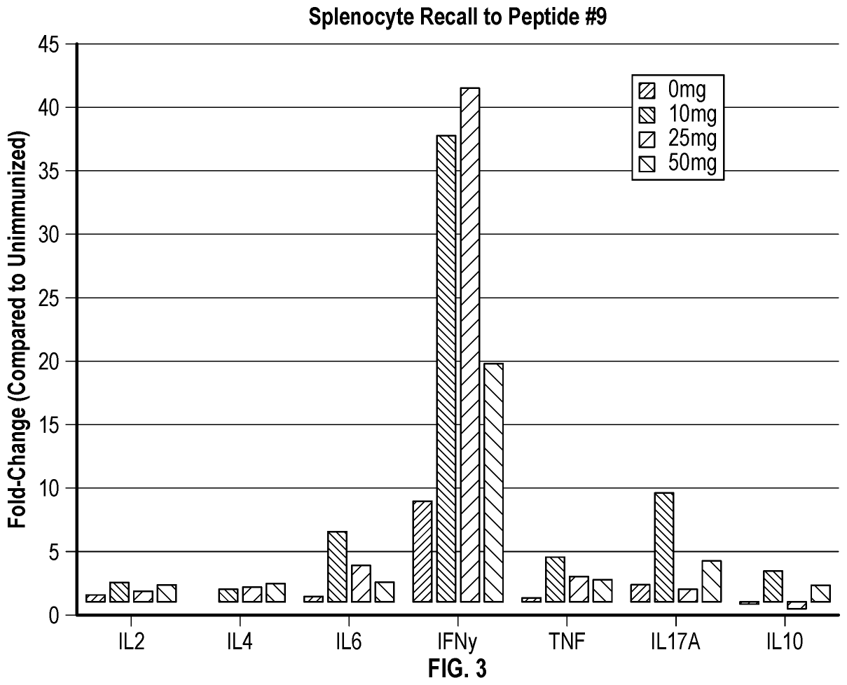

In FIG. 3, Mice were immunized with peptide #9 and on day 3, the cytokine response was measured in response to peptide #9 given in culture of spleen cells from immunized mice. Here it is clear that the response to IL6, IFNg, TNF, IL17, and IL 10 was DPCA dose-dependent. IFNg was most dramatically affected. The bar colors represent 0 ug/mouse of DPCA (blue), 10 ug /ouse of DPCA (orange), 25 ug/mouse of DPCA (grey), and 50 ug /ouse of DPCA (yellow).

These data show that 1,4-DPCA-PEG enhances antigen-specific T cell responses. This is true for both chosen peptides used. Additionally, there is increased polyfunctionality with particular high levels of expression of inflammatory cytokines. The lowest dose used (10 ug is optimal in almost all cases, orange bars).

### Example 2: 1,4DPCA Effect at High Doses

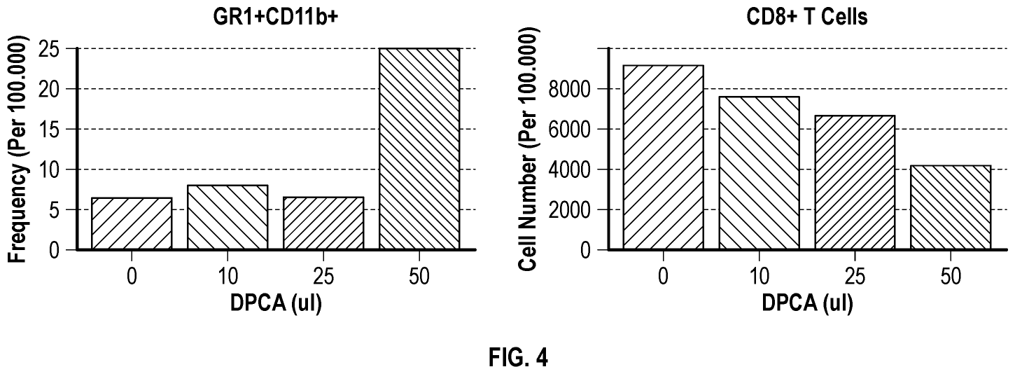

To explore what is happening at higher DPCA doses and why they may be inhibitory, we tested the effects of different concentrations of DPCA on myeloid (GR1+CD11b+) cells and lymphoid (CD8+) cells. (FIG. 4) We found that the number of CD8+T cells were reduced. Also, the number of GR1+CD11b+myeloid cells is much higher. Within this population of myeloid cells are cells that can suppress T cells. This could explain why higher doses of DPCA leads to poorer T cell responses as seen in FIGS. 2 and 3.

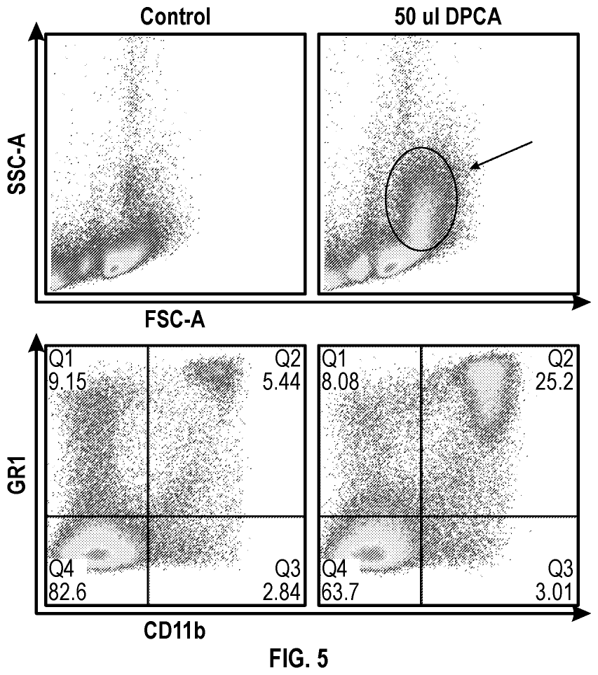

Additionally, cells were immunized with 50 ug of DPCA and compared to an untreated control. The number of cell in each was determined by FACS analysis. In FIG. 5 FACS analysis shows that immunization with 50 ug of DPCA showed an increase number of large cells. (See upper panels showing scatter plots with an increased number of cells in treated cells). An analysis of myeloid cell markers GR1 and CD11b was determined in the control and DPCA treated cells. FIG. 5 indicates that treated mice show a large population of myeloid cells when compared to an untreated control. (See lower panels)

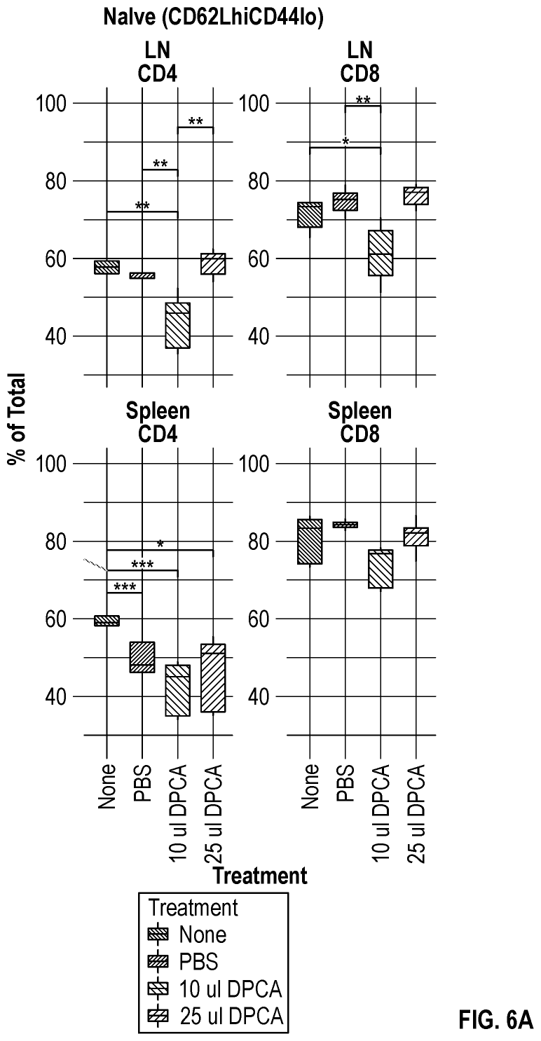

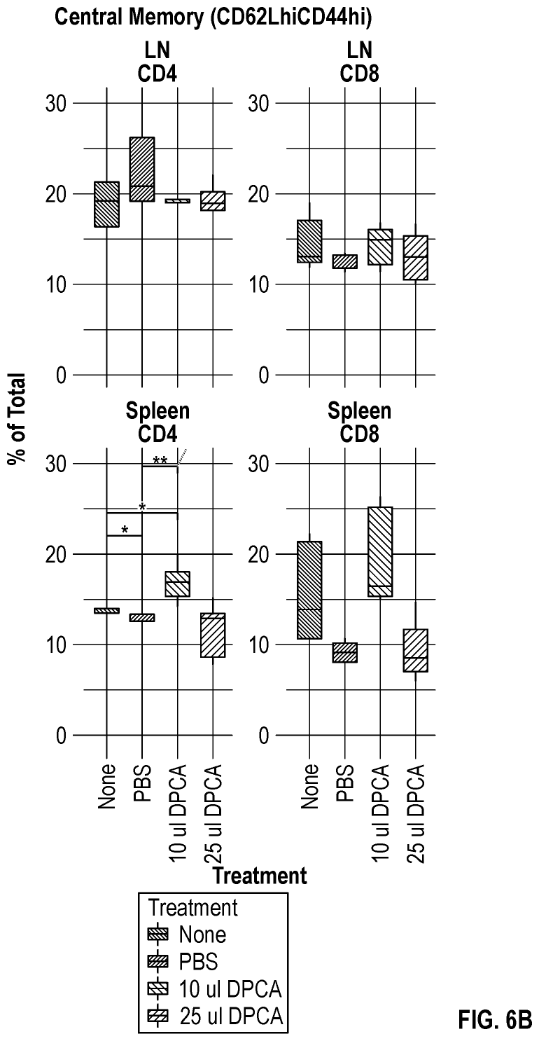

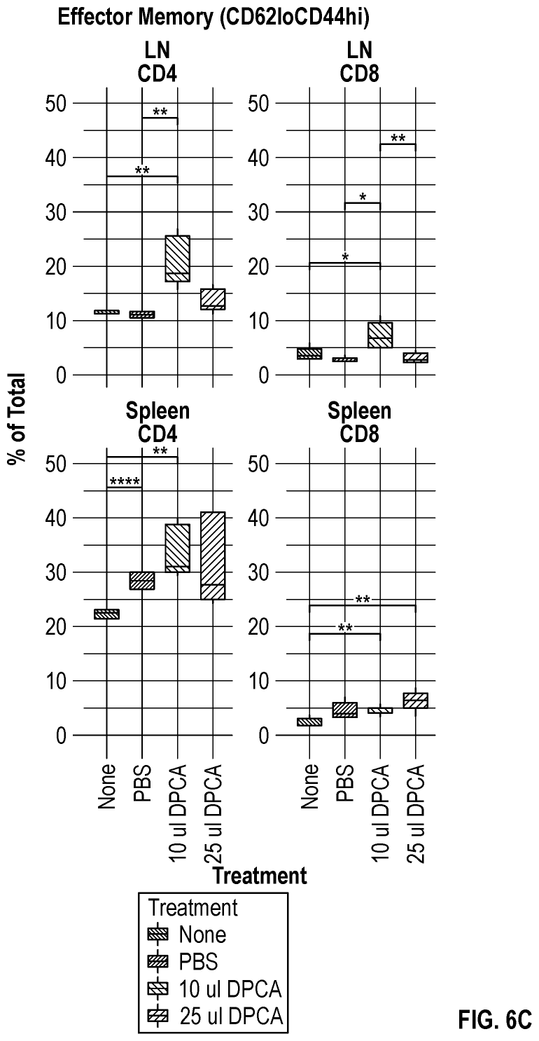

Next, three populations of T cells (Naïve, central memory, and effector T cells) from lymph nodes and spleens were treated with peptide 9 and 10 ug of DPCA or 25 ug of DPCA. An untreated control group and a control group treated with PBS were also tested. FIG. 6 shows the numbers of CD4 and CD8 cells in each group. Treatment with DPCA generally shows increased numbers of memory cells but lower numbers in naïve cells.

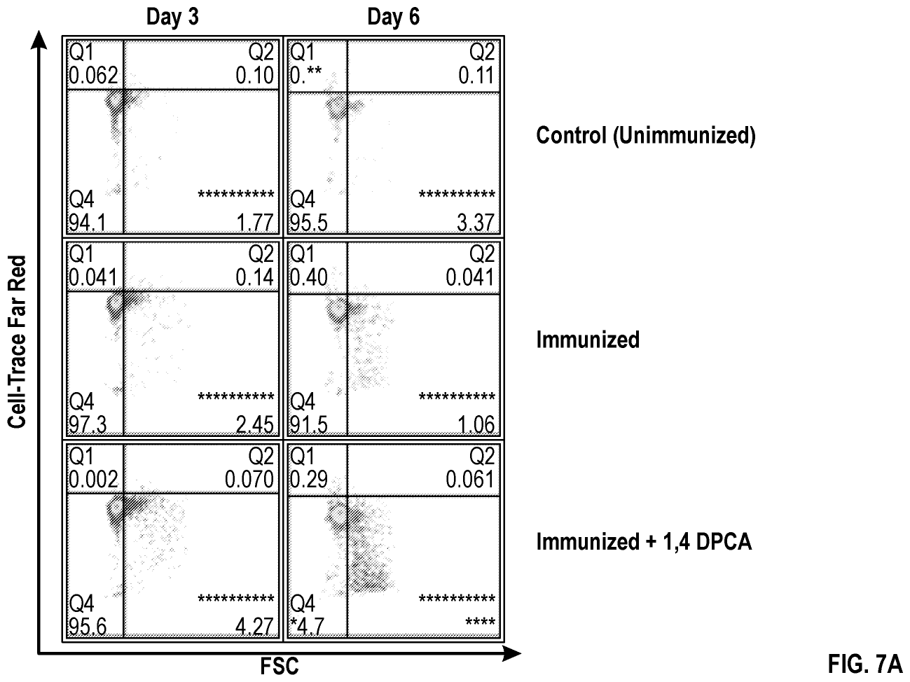

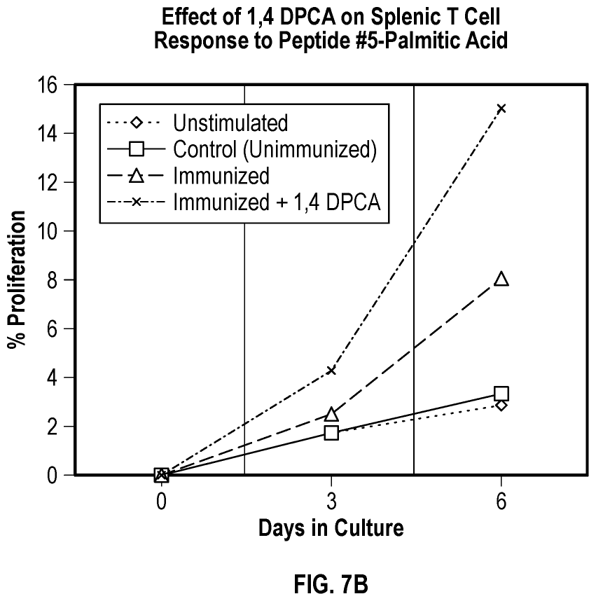

Mice were now immunized with peptide 5 and T cells analyzed for proliferation and by FACS analysis. (FIG. 7) Mice were immunized subcutaneously with peptide #5-palmitic acid CFA mixture with and without 10 ug of 1,4-DPCA. 14 days later, splenic T cells were isolated and co-cultured with irradiated feeders in a ratio of 400k:100k to assess recall responses to peptide #5-palmitic acid. T cells from unimmunized mice were used as control. Only T cells were stained with CTFR proliferation dye in order to ensure detection of specific T cell responses.

In upper FIG. 7 is the FACS flow cytometry plot, where the labelled cells are T cells. In the immunized and immunized+DPCA regions, the cells move to the right quadrant and then down (shifting because of increased proliferation). Below this, is the analyzed proliferation data, where the yellow line is immunized+10 ug of DPCA was given to mice, the grey line is immunized only, the orange line is control, unimmunized but stimulated in culture, and the blue line is control, unstimulated in culture.

In conclusion, we have shown using two randomly chosen peptides from the Spike protein of COVID 19, that antigen stimulation after immunization is greatly enhanced by DPCA-PEG, both proliferation and cytokine production. We have also shown that DPCA-PEG can enhance bone marrow-derived cell migration into the spleen and lymph node which we assume consists of immune cells.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### EMBODIMENTS

Embodiment 1. A method for enhancing a patient's immune response to an immune stimulatory composition, the method comprising administering an immune stimulatory composition and at least one agent that affects metabolic reprogramming.
Embodiment 2. The method of embodiment 1, wherein the agent that affects metabolic reprogramming is selected from:
   a. an inhibitor of the proline hydroxylase (PHD) pathway;
   b. an inhibitor of a p21 kinase; or
   c. an agonist of HIF-1α.
Embodiment 3. The method of embodiment 2, wherein the agonist of HIF-1α is a modulator of a protein in the HIF regulatory pathway.
Embodiment 4. The method of any one of embodiments 1 to 3, wherein the agent at least transiently upregulates, increases, or stabilizes HIF1.
Embodiment 5. The method of any one of the preceding embodiments, wherein the agent is a small molecule.
Embodiment 6. The method of any one of the preceding embodiments, wherein the agent is a protein, peptide, or nucleic acid sequence.
Embodiment 7. The method of embodiment 6, wherein the nucleic acid sequence is an siRNA or a miRNA.
Embodiment 8. The method of any one of the preceding embodiments, wherein the agent is a PHD inhibitor or prodrug thereof.
Embodiment 9. The method of embodiment 8, wherein the PHD inhibitor is 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), Hydralazine, P80D6 or P7D3.
Embodiment 10. The method of embodiment 9, wherein the PHD inhibitor is 1,4-DPCA.
Embodiment 11. The method of embodiment 9, wherein the PHD inhibitor is at least one poly(alkaline oxide) coupled prodrug of 1,4-DPCA.
Embodiment 12. The method of embodiment 11, wherein the PHD inhibitor is at least a first poly(alkaline oxide) coupled prodrug of 1,4-DPCA and a second poly(alkaline oxide) coupled prodrug of 1,4-DPCA.
Embodiment 13. The method of embodiment 12, wherein the first poly(alkaline oxide) coupled prodrug of 1,4-DPCA has a high molecular weight, and the second poly(alkaline oxide) coupled prodrug of 1,4-DPCA has a low molecular weight.
Embodiment 14. The method of embodiment 13, wherein the first poly(alkaline oxide) coupled prodrug of 1,4-DPCA is P80D6 and the second poly(alkaline oxide) coupled prodrug of 1,4-DPCA is P7D3.
Embodiment 15. The method of any one of embodiments 13-14, wherein the first and second poly(alkaline oxide) coupled prodrugs of 1,4-DPCA are in a ratio of approximately 45:55-55:45.
Embodiment 16. The method of any one of embodiments 13-15, the first and second poly(alkaline oxide) coupled prodrugs of 1,4-DPCAare in a ratio of approximately 47:53.
Embodiment 17. The method of embodiment 1, wherein the HIF-la agonist is a protease-activated receptor 1 (PAR-1) agonist.
Embodiment 18. A method of treating cancer, the method comprising administering a PHD inhibitor with an anticancer therapy.
Embodiment 19. The method of embodiment 17, wherein the anticancer therapy has vaccine-like effects.
Embodiment 20. The method of embodiment 17 or embodiment 18, wherein the anticancer therapy is selected from chemotherapy, radiotherapy, cryotherapy, or another tumor ablative modality.
Embodiment 21. A method of enhancing an immune response, the method comprising administering a PHD inhibitor in combination with a vaccine, wherein the treatment increases the strength and/or potency of the immune response when compared to administration of a vaccine without a PHD inhibitor.
Embodiment 22. The method of embodiment 21, wherein the vaccine is directed towards an infectious disease.
Embodiment 23. The method of embodiment 21, wherein the vaccine is directed towards a SARS-CoV2 Spike protein epitope.
Embodiment 24. The method of any one of the preceding embodiments, wherein the patient is elderly or has an attenuated immune response to the immune stimulatory composition alone when compared to a healthy patient.
Embodiment 25. The method of any one of the preceding embodiments, wherein the patient has an attenuated immune response to the immune stimulatory composition alone when compared to a healthy patient.
Embodiment 26. The method of any one of the preceding embodiments, wherein the immune stimulatory composition and/or agent are administered in a liposomal formulation.
Embodiment 27. The method of any one of the preceding embodiments, wherein the immune stimulatory composition and/or agent are administered in a lipid nanoparticle formulation.
Embodiment 28. The method of any one of the preceding embodiments, wherein the immune stimulatory composition and/or agent are administered via a subcutaneous or intramuscular injection.
Embodiment 29. The method of any one of the preceding embodiments, wherein the immune stimulatory composition and/or agent are administered orally or topically at a mucosal site.
Embodiment 30. The method of any one of the preceding embodiments, wherein the agent is administered at a concentration of 10-20 μM.
Embodiment 31. A composition comprising a vaccine and an adjuvant selected from at least one agent that affects metabolic reprogramming.
Embodiment 32. The composition of embodiment 31, wherein the agent that affects metabolic reprogramming is selected from:
   a. an inhibitor of the proline hydroxylase (PHD) pathway;
   b. an inhibitor of a p21 kinase; or
   c. an agonist of HIF-1α.
Embodiment 33. The composition of embodiment 32, wherein the agonist of HIF-1α is a modulator of a protein in the HIF regulatory pathway.
Embodiment 34. The composition of any one of embodiments 31 to 33, wherein the agent at least transiently upregulates, increases, or stabilizes HIF1.
Embodiment 35. The composition of any one of embodiments 31 to 34, wherein the agent is a small molecule.
Embodiment 36. The composition of any one of embodiments 31 to 35, wherein the agent is a protein, peptide, or nucleic acid sequence.
Embodiment 37. The composition of embodiment 36, wherein the nucleic acid sequence is an siRNA or a miRNA.
Embodiment 38. The composition of any one of embodiments 32-37, wherein the agent is a PHD inhibitor or prodrug thereof.
Embodiment 39. The composition of embodiment 38, wherein the PHD inhibitor is 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), or Hydralazine.
Embodiment 40. The composition of embodiment 39, wherein the PHD inhibitor is 1,4-DPCA.
Embodiment 41. The composition of embodiment 39, wherein the PHD inhibitor is at least one poly(alkaline oxide) coupled prodrug of 1,4-DPCA.
Embodiment 42. The composition of embodiment 39, wherein the PHD inhibitor is at least a first poly(alkaline oxide) coupled prodrug of 1,4-DPCA and a second poly(alkaline oxide) coupled prodrug of 1,4-DPCA.
Embodiment 43. The composition of embodiment 40, wherein the first poly(alkaline oxide) coupled prodrug of 1,4-DPCA has a high molecular weight, and the second poly(alkaline oxide) coupled prodrug of 1,4-DPCA has a low molecular weight.
Embodiment 44. The composition of embodiment 41, wherein the first poly(alkaline oxide) coupled prodrug of 1,4-DPCA is P80D6 and the second poly(alkaline oxide) coupled prodrug of 1,4-DPCA is P7D3
Embodiment 45. The composition of any one of embodiments 43-44, wherein the first and second poly(alkaline oxide) coupled prodrugs of 1,4-DPCA are in a ratio of approximately 45:55-55:45.
Embodiment 46. The composition of any one of embodiments 43-45, the first and second poly(alkaline oxide) coupled prodrugs of 1,4-DPCAare in a ratio of approximately 47:53.
Embodiment 47. The composition of embodiment 32, wherein the HIF-la agonist is a protease-activated receptor 1 (PAR 1) agonist.

1. A method for enhancing a patient's immune response to an immune stimulatory composition, the method comprising administering an immune stimulatory composition and at least one agent that affects metabolic reprogramming.

2. The method of claim 1, wherein the agent that affects metabolic reprogramming is selected from:
a. an inhibitor of the proline hydroxylase (PHD) pathway;
b. an inhibitor of a p21 kinase; or
c. an agonist of HIF-1α.

3. The method of claim 2, wherein the agonist of HIF-1α is a modulator of a protein in the HIF regulatory pathway.

4. The method of claim 1, wherein the agent at least transiently upregulates, increases, or stabilizes HIF1.

5. The method of claim 1, wherein the agent is
a) a small molecule;
b) a protein, peptide, or nucleic acid sequence; or
c) an siRNA or miRNA.

6.-7. (canceled)

8. The method of claim 2, wherein the agent is a PHD inhibitor or prodrug thereof.

9. The method of claim 8, wherein the PHD inhibitor is 1, 4-dihydrophenothrolin-4-one-3-carboxylic acid (1,4-DPCA), a poly(alkaline oxide) coupled prodrug of 1,4-DPCA, Fibrogen (FG) 4592, Ciclopirox, Dibenzoylmethane; Deferoximide (deferoxamine), or Hydralazine.

10.-11. (canceled)

12. The method of claim 9, wherein the PHD inhibitor is at least a first poly(alkaline oxide) coupled prodrug of 1,4-DPCA and a second poly(alkaline oxide) coupled prodrug of 1,4-DPCA.

13. The method of claim 12, wherein the first poly(alkaline oxide) coupled prodrug of 1,4-DPCA has a high molecular weight, and the second poly(alkaline oxide) coupled prodrug of 1,4-DPCA has a low molecular weight.

14. The method of claim 13, wherein the first poly(alkaline oxide) coupled prodrug of 1,4-DPCA is P80D6 and the second poly(alkaline oxide) coupled prodrug of 1,4-DPCA is P7D3.

15. The method of claim 13, wherein the first and second poly(alkaline oxide) coupled prodrugs of 1,4-DPCA are in a ratio of approximately 45:55-55:45.

16. The method of claim 15, the first and second poly(alkaline oxide) coupled prodrugs of 1,4-DPCAare in a ratio of approximately 47:53.

17.-20. (canceled)

21. A method of enhancing an immune response, the method comprising administering a PHD inhibitor in combination with a vaccine, wherein the treatment increases the strength and/or potency of the immune response when compared to administration of a vaccine without a PHD inhibitor.

22. The method of claim 21, wherein the vaccine is directed towards an infectious disease or a SARS-CoV2 Spike protein epitope.

23. (canceled)

24. The method of claim 1, wherein the patient is elderly or has an attenuated immune response to the immune stimulatory composition alone when compared to a healthy patient.

25. The method of claim 1, wherein the patient has an attenuated immune response to the immune stimulatory composition alone when compared to a healthy patient.

26.-29. (canceled)

30. The method of claim 1, wherein the agent is administered at a concentration of 10-20 μM.

31. A composition comprising a vaccine and an adjuvant selected from at least one agent that affects metabolic reprogramming.

32.-37. (canceled)

38. The composition of claim 31, wherein the agent is a PHD inhibitor or prodrug thereof wherein said PHD inhibitor is at least a first poly(alkaline oxide) coupled prodrug of 1,4-DPCA and a second poly(alkaline oxide) coupled prodrug of 1,4-DPCA.

39.-43. (canceled)

44. The composition of claim 38, wherein the first poly(alkaline oxide) coupled prodrug of 1,4-DPCA is P80D6 and the second poly(alkaline oxide) coupled prodrug of 1,4-DPCA is P7D3.

45.-47. (canceled)
